(19) United States
(12) Patent Application Publication
(10) Pub. No.: US 2010/0125156 A1
(43) Pub. Date: May 20, 2010

(54) Condensation reactions for polyols

(76) Inventor: Kevin W. Smith, Magnolia, TX (US)

Correspondence Address: William L. Krayer, 1771 Helen Drive, Pittsburgh, PA, 15216, US

(21) Appl. No.: 12/590,688

(22) Filed: Nov. 12, 2009

Related U.S. Application Data
(60) Provisional application No. 61/199,235, filed on Nov. 14, 2008.

(51) Int. Cl. C07C 29/34

(52) U.S. Cl. 568/852

(57) ABSTRACT

Polyols having three or more OH groups (especially glycerol) are condensed to form higher molecular weight polyols by heating them in a cavitation or other heating device and separating, by evaporation, the water formed in the reaction, the separation being preferably assisted by the application of a subatmospheric pressure. An appropriate balance between the reactants and the water formed is maintained by recycling and the introduction of additional lower molecular weight reactants in a continuous process. The polyalcohols, particularly the polyglycerine, are useful in shale stabilization in the treatment of wells for hydrocarbon recovery.

### RELATED APPLICATION

This application claims the full benefit of Provisional application 61/100235 filed Nov. 14, 2008.

### TECHNICAL FIELD

Polyols having three or more OH groups (especially glycerol) are condensed to form higher molecular weight polyols by heating them in a cavitation device and separating, by evaporation, the water formed in the reaction, the separation being assisted by the cavitation device and the application of a subatmospheric pressure. An appropriate balance between the reactants and the water formed is maintained by recycling and the introduction of additional lower molecular weight reactants in a continuous process. The polyalcohols are useful in shale stabilization in the treatment of wells for hydrocarbon recovery.

### BACKGROUND OF THE INVENTION

The autocondensation of polyols to make higher molecular weight polyols is known. See the descriptions of the processes in Peterson U.S. Pat. No. 4,780,220, Cowan U.S. Pat. No. 5,337,824, Hale et al U.S. Pat. No. 5,076,373, and Blytas U.S. Pat. No. 5,371,244. The specific structures of the components of the reaction products can be rather complex; for example, cyclic ethers are commonly obtained. The basic functionality of the OH groups, however, together with the properties such as viscosity imparted by the higher molecular weight, have been recognized for various uses, as described in the just recited patents.

While certain methods of making such compositions are described in the above recited patents, the effects of temperature, mixing, and removal of water remain difficult to control and maintain, to achieve desired properties.

Reference is made below to glycerine. This is also known as 1,2,3-trihydroxypropane and 1,2,3-propanetriol as well as glycerol. Polyglycerine is technically glycerol homopolymer, but the name has also been applied to any condensation reaction product of glycerol. I use the term polyglycerine here to include materials made by autocondensing glycerol, which may include various dimers, trimers and other higher molecular weight compounds, including materials which are not entirely linear, as will appear below.

### SUMMARY OF THE INVENTION

I use a cavitation device or other heating device as a reactor to heat lower molecular weight polyols, effecting their autocondensation, and to remove the water of condensation formed in the reaction. Evaporation of the water may be encouraged by drawing a vacuum directly on the outlet of the cavitation or other heating device, through a condenser, or overhead from a flash tank. The process is particularly applicable to the condensation of glycerol with itself.

My invention is particularly useful when practiced as a substantially continuous process for making higher molecular weight products such as polyglycerine including little or no free water.

### BRIEF DESCRIPTION OF THE DRAWINGS

FIGS. 1a and 1b depict a cavitation device of a type useful in my invention.

FIG. 2 is a basic flow diagram of my process utilizing a cavitation device.

### DETAILED DESCRIPTION OF THE INVENTION

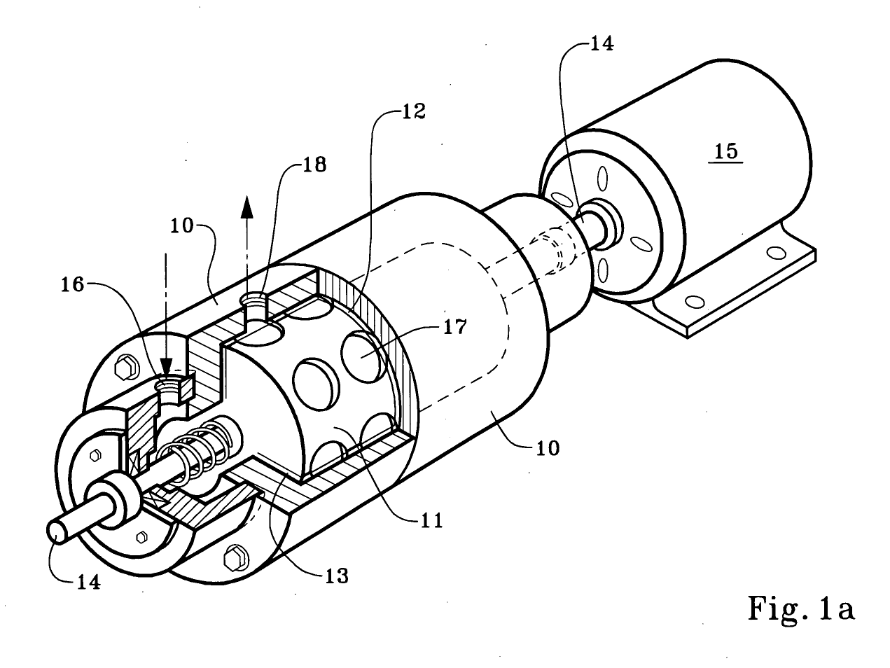

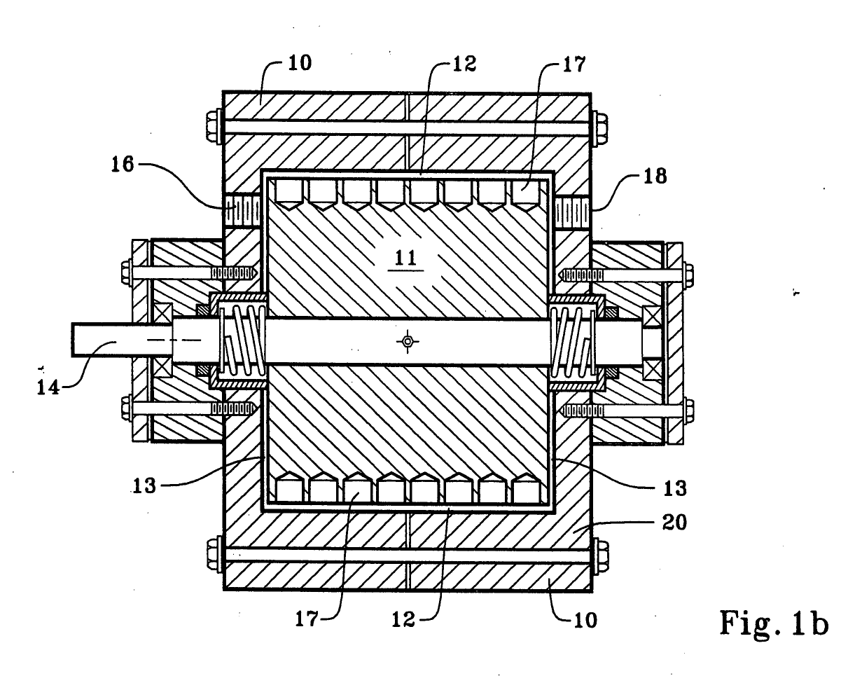

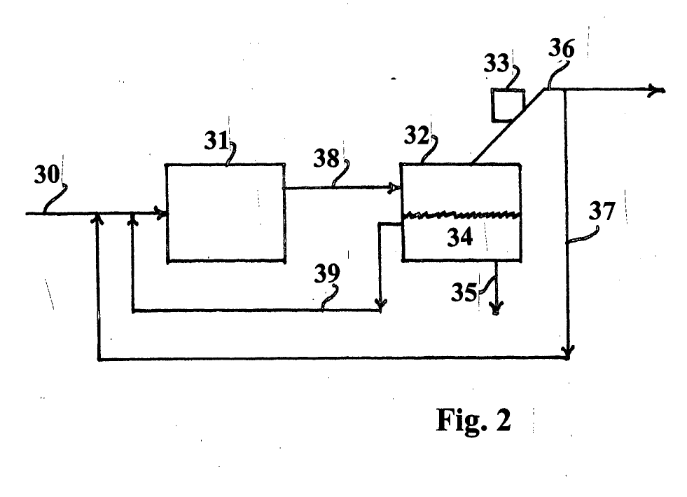

Referring now to FIGS. 1a and 1b, FIGS. 1a and 1b show two slightly different variations, and views, of a cavitation device useful for effecting the autocondensation of polyols. FIGS. 1a and 1b are taken from FIGS. 1 and 2 of Griggs U.S. Pat. No. 5,188,090, which is specifically incorporated herein by reference in its entirety along with related U.S. Pat. Nos. 5,385,298, 5,957,122, and 6,627,784, all describing devices manufactured and sold by Hydro Dynamics, Inc., of Rome, Ga. In recent years, Hydro Dynamics, Inc. has adopted the trademark “Shockwave Power Reactor” for its cavitation devices, and I sometimes use the term SPR herein to describe the products of this company and other cavitation devices that can be used in my invention.

A housing 10 in FIGS. 1a and 1b encloses cylindrical rotor 11 leaving only a small clearance 12 around its curved surface and clearance 13 at the ends. The rotor 11 is mounted on a shaft 14 turned by motor 15. Cavities 17 are drilled or otherwise cut into the surface of rotor 11. As explained in the Griggs patent, other irregularities, such as shallow lips around the cavities 17, may be placed on the surface of the rotor 11. Some of the cavities 17 may be drilled at an angle other than perpendicular to the surface of rotor 11—for example, at a 15 degree angle. Liquid (fluid)—in the case of the present invention, one or more low molecular weight polyols,—is introduced through port 16 under pressure and enters clearances 13 and 12. As the fluid passes from port 16 to clearance 13 to clearance 12 and out exit 18 while the rotor 11 is turning, areas of vacuum are generated and heat is generated within the fluid from its own turbulence, expansion and compression (shock waves). As explained at column 2 lines 61 et seq in the Griggs U.S. Pat. No. 5,188,090, “(T)he depth, diameter and orientation of (the cavities) may be adjusted in dimension to optimize efficiency and effectiveness of (the cavitation device) for heating various fluids, and to optimize operation, efficiency, and effectiveness . . . with respect to particular fluid temperatures, pressures and flow rates, as they relate to rotational speed of (the rotor 11).” Smaller or larger clearances may be provided (col. 3, lines 9-14). Also the interior surface of the housing 10 may be smooth with no irregularities or may be serrated, feature holes or bores or other irregularities as desired to increase efficiency and effectiveness for particular fluids, flow rates and rotational speeds of the rotor 11. (col. 3, lines 23-29) Rotational velocity may be on the order of 5000 rpm (col 4 line 13). The diameter of the exhaust ports 18 may be varied also depending on the particular polyol feed and the desired outcome. Note that the position of exit port 18 is somewhat different in FIGS. 1a and 1b; likewise the position of entrance port 16 differs in the two versions and may also be varied to achieve different effects in the flow pattern within the SPR. Port 16 may be referred to herein as a reaction mixture inlet and one or more exit ports 18 may be referred to herein as a reaction product outlet.

Another variation which can lend versatility to the SPR is to design the opposing surfaces of housing 10 and rotor 11 to be somewhat conical, and to provide a means for adjusting the position of the rotor within the housing so as to increase or decrease the width of the clearance 12. This can allow for variations in the viscosity of the fluid, to reduce the shearing effect if desired (by increasing the width of clearance 12), to vary the velocity of the rotor as a function of the fluid's viscosity, or for any other reason.

Definition: I use the term “cavitation device,” or “SPR,” to mean and include any device which will cause bubbles or pockets of partial vacuum to form within the liquid it processes. The bubbles or pockets of partial vacuum have also been described as areas within the liquid which have reached the vapor pressure of the liquid. The turbulence and/or impact, which may be called a shock wave, caused by the implosion imparts thermal energy to the liquid, which, in the case of water, may readily reach boiling temperatures. The bubbles or pockets of partial vacuum are typically created by flowing the liquid through narrow passages which present side depressions, cavities, pockets, apertures, or dead-end holes to the flowing liquid; hence the term “cavitation effect” is frequently applied, and devices known as “cavitation pumps” or “cavitation regenerators” are included in my definition. Steam or water vapor generated in the cavitation device can be separated from the remaining, now concentrated, water and more or less polymerized polyols.

The term “cavitation device” includes not only all the devices described in the above itemized U.S. Pat. Nos. 5,385,298, 5,957,122 6,627,784 and 5,188,090 but also any of the devices described by Sajewski in U.S. Pat. Nos. 5,183,513, 5,184,576, and 5,239,948, Wyszomirski in U.S. Pat. No. 3,198,191, Selivanov in U.S. Pat. No. 6,016,798, Thoma in U.S. Pat. Nos. 7,089,886, 6,976,486, 6,959,669, 6,910,448, and 6,823,820, Crosta et al in U.S. Pat. No. 6,595,759, Giebeler et al in U.S. Pat. Nos. 5,931,153 and 6,164,274, Huffman in U.S. Pat. No. 5,419,306, Archibald et al in U.S. Pat. No. 6,596,178 and other similar devices which employ a shearing effect between two close surfaces, at least one of which is moving, such as a rotor, and at least one of which has cavities of various designs in its surface as explained above.

Operation of the SPR (cavitation device) is as follows. A shearing stress is created in the fluid as it passes into the narrow clearance 12 between the rotor 11 and the housing 10. The solution quickly encounters the cavities 17 in the rotor 11, and tends to fill the cavities, but the centrifugal force of the rotation tends to throw the liquid back out of the cavity. The shearing stress and cavitation phenomona heat the liquid essentially without using a heat transfer surface.

A method of utilizing the cavitation device in my invention is shown in FIG. 2. As seen in FIG. 2, a low molecular weight polyol feed is sent substantially continuously through line 30 to the cavitation device 31, where it is subjected to cavitation and therefore heated and mixed intimately as explained above, effecting a condensation reaction among the OH groups to a degree determined by the temperature and other conditions in the cavitation device. The polyol feed may be, for example, glycerine (glycerol), a mixture of glycerine and other low molecular weight polyols, or water mixed with either glycerine or a mixture of glycerine with other low molecular weight polyols. The heated reaction mixture is sent through line 38 to a flash tank 32 which is subject to a vacuum by vacuum pump 33. The subatmospheric pressure effected by vacuum pump 33 extends to the cavitation device 31, thus enabling evaporation of water at a temperature below atmospheric boiling. Volatile polyols may also remain in gaseous form, but higher molecular weight polyols are separated as a liquid mixture 34 in the bottom of flash tank 32 and can be collected by means of drain 35. The liquid mixture 34 or the lighter portions thereof may be recycled to cavitation device 31 by way of line 39. Vapor in line 36 may be separated by any suitable condensation or other means into a substantially aqueous condensate and a substantially polyol condensate, which may be returned to the cavitation device through line 37. Such a substantially polyol condensate may comprise unreacted glycerine.

I do not intend to be limited to a flash tank for separating water from the reaction product. Any suitable gas-liquid separating device or method may be used. Where the lower polyol(s) or glycerine is introduced substantially continuously, and the temperature, pressure, and liquid/vapor separation is substantially continuous, my invention is not limited to the use of a cavitation device. Any suitable heating device may be used where cavitation device 31 is illustrated in FIG. 2.

The reaction may be enhanced by introduction to the cavitation or other heating device of a catalyst such as sodium hydroxide in an amount effective to enhance the condensation reaction.

The product collected from drain 35 may be called polyglycerine, particularly where glycerine (glycerol) is the only reactant, but it may contain substantial quantities of a dimer of glycerine (diglycerine), which may be linear or cyclic, a trimer of glycerine (triglycerine), which may be linear or cyclic, and higher combinations such as tetraglycerine, pentaglycerine and heavier polyglycerine, any of which may contain cyclic ether groups. Adjusting the temperature, pressure, and recycle of lower molecular weight materials will enable the operator to achieve various desired average molecular weights and/or high concentrations of particular components.

The following further descriptions of the reaction product components are adapted from Hale et al U.S. Pat. No. 5,076,373, which is specifically incorporated herein by reference in its entirety. My process makes acyclic polyols. Common examples are polyglycerines of the formula formula H—(OCH₂CHOH—CH₂)ₙ—OH where n is an integer from 3 to 6. Among acyclic polyols, preferred are those having at least 3 carbon atoms and 2 hydroxyl groups but no more than 80 carbon atoms and 60 hydroxyl groups. More preferably, the acyclic polyols of the invention have at least 9 carbon atoms and at least 5 hydroxyl groups but no more than 50 carbon atoms and 40 hydroxyl groups.

The invention also makes monoalicylicpolyols. Among monoalicylicpolyols, preferred are those having 5 to 30 carbon atoms and 2 to 10 hydroxyl groups.

Nonlimiting examples of other compounds include monomers, oligomers and telomers of polyhydric alcohols (or their precursors, or combinations thereof) such as telomers of glycerol such as diglycerols, triglycerols, tetraglycerols, pentaglycerols, and hexaglycerols, mixtures of glycerol and telomers of glycerol such as diglycerol and triglycerols, and mixtures of telomers of glycerol. The reaction mixture may commonly include six-membered cyclic diether groups.

The cavitation device is particularly useful in my process because of its excellent mixing abilities and the ability to impart high temperatures to the feedstock rather quickly; it also readily facilitates recycle of a portion of the device's output. The recycle stream can be adjusted to include lighter molecules that may be in the gaseous phase, leading to a higher molecular weight product than might be the case otherwise. However, any suitable heating device may be used for my continuous process yielding polyglycerine having less then 5%, preferably less than 2% water by weight.

My invention therefore includes a method of polymerizing glycerine comprising substantially continuously autocondensing said glycerine in a heating device under a temperature and pressure adequate to continuously autocondense said glycerine while vaporizing water given off thereby, and substantially continuously removing said water vapor from said heating device. The heating device may be a cavitation device, but need not be.

My invention also includes a method of making polyglycerine comprising (a) introducing glycerine to a heating device, (b) heating said glycerine in said heating device, (c) removing liquid product and vapor product from said heating device, (d) separating said vapor product from said liquid product, and (e) recovering said polyglycerine as said liquid product, wherein said liquid product contains less than 5% by weight free water. The polyglycerine product preferably has less than 5% by weight free water, and more preferably less than 2% by weight free water.

In addition, my invention includes a method of conducting an autocondensation reaction of at least one low molecular weight polyol comprising heating said at least one low molecular weight polyol in a cavitation device to make a reaction mixture, and removing water from said reaction mixture.

1. Method of conducting an autocondensation reaction of at least one low molecular weight polyol comprising heating said at least one low molecular weight polyol in a cavitation device to make a reaction mixture, and removing water from said reaction mixture.

2. Method of claim 1 which is substantially continuous.

3. Method of claim 1 wherein said low molecular weight polyol comprises glycerine.

4. Method of claim 1 wherein said reaction mixture comprises diglycerine and triglycerine.

5. Method of claim 1 including introducing a condensation reaction catalyst to said cavitation device in an amount effective to enhance said condensation reaction.

6. Method of claim 1 wherein water is removed from said reaction mixture by evaporation and extraction under a subatmospheric pressure.

7. Method of claim 6 wherein said cavitation device has a reaction mixture inlet and a reaction product outlet, and wherein said subatmospheric pressure is applied at said reaction product outlet.

8. Method of claim 6 wherein a high molecular weight polyol is collected after said evaporation.

9. Method of claim 6 including recycling at least some low molecular weight polyol obtained in said evaporation.

10. Method of polymerizing glycerine comprising substantially continuously autocondensing said glycerine in a heating device under temperatures and pressures adequate to continuously autocondense said glycerine while vaporizing water given off thereby, and substantially continuously removing said water vapor from said heating device.

11. Method of claim 10 including controlling the temperature and pressure in said heating device to obtain a product comprising H—(OCH₂CHOH—CH₂)ₙ—OH where n is an integer from 3 to 6.

12. Method of claim 10 including separating water from said glycerine autocondensed thereby in a gas-liquid separation device.

13. Method of making polyglycerine comprising (a) introducing glycerine to a heating device, (b) heating said glycerine in said heating device, (c) removing liquid product and vapor product from said heating device, (d) separating said vapor product from said liquid product, and (e) recovering said polyglycerine as said liquid product, wherein said liquid product contains less than 5% by weight free water.

14. Method of claim 13 wherein steps (a), (b), (c), (d), and (e) are substantially continuous.

15. Method of claim 13 including condensing water from said vapor product.

16. Method of claim 13 including recycling at least some low molecular weight polyol from said vapor product to said heating device.

17. Method of claim 13 including recycling at least some liquid product to said heating device.

18. Method of claim 15 wherein said polyglycerine liquid product contains less than 2% by weight free water.

19. Method of claim 13 conducted in the presence of a condensation catalyst.

20. Method of claim 13 wherein step (d) is performed in a flash tank.
